(19) United States
(12) Patent Application Publication
(10) Pub. No.: US 2024/0335683 A1
(43) Pub. Date: Oct. 10, 2024

(54) METHOD FOR BLEACHING THE HAIR

(71) Applicant: L'OREAL, Paris (FR)

(72) Inventors: Valeria NUZZO, Saint-Ouen (FR); Gabin VIC, Clichy (FR); Melissa LIAUZUN, Chevilly-Larue (FR)

(21) Appl. No.: 18/681,449

(22) PCT Filed: Oct. 27, 2022
(86) PCT No.: PCT/EP2022/080083

§ 371 Date: Feb. 5, 2024

(30) Foreign Application Priority Data
Oct. 29, 2021 (FR) FR2111519

(51) Int. Cl. A61Q 5/08; A61K 8/23

(52) U.S. Cl. CPC A61Q 5/08; A61K 8/23; A61K 2800/81

(57) ABSTRACT

A cosmetic method for bleaching the hair, including a) applying a bleaching composition comprising one or more persulfates to at least one lock of hair, b) exposing the hair impregnated with the bleaching composition to microwave radiation.

### TECHNICAL FIELD

The present invention relates to methods for bleaching the hair.

### PRIOR ART

Numerous techniques for bleaching the hair have already been proposed.

It is known in particular to use bleaching compositions containing persulfates, with or without aqueous hydrogen peroxide solution. The use of such compositions requires long leave-on times and high concentrations of oxidizing agents, and this is not always desirable both from the harmlessness point of view and from the point of view of maintaining the integrity of the hair fibers.

In the field of surface maintenance, it is known to use microwave radiation to increase the action of detergents. In particular, the patent applications WO 98/03621, WO 98/03622, WO 98/03623, WO 99/25802 and WO 99/37745 describe treatments for hard surfaces combining exposure to microwave radiation with detergents containing whitening agents.

In the field of shaping the head of hair, the patent applications DE 3148 538, US 2006/0108358, US 2010/0006116 and US 2007/056860 disclose the direct use of microwave radiation on the hair.

The documents US 2006/042649 and U.S. Pat. No. 6,079,422 describe heatable curlers that can be placed in a microwave oven before being used to wind the hair. In these documents, it is the aspect of heat generation by the microwaves that is used, but these microwaves are not directly placed in contact with the hair.

The patent application WO 2013/183021 discloses a method for treating keratin fibers comprising the application of a shaping composition followed by the application of a mechanical tension and then by exposure to microwave radiation.

The patent applications WO 2011/141882 and FR 3 062 786 describe hair treatment methods with a step of placing under mechanical tension and a step of exposure to microwave radiation.

The patent application WO 2019/122850 discloses a method for coloring the hair using a dye composition and simultaneous treatment with radio wave radiation with a frequency of between 1 MHz and 300 MHz.

Consequently, in light of these documents, there is still a need to improve the methods for bleaching the hair, by increasing their efficacy while at the same time optimally preserving the hair fiber.

### DISCLOSURE OF THE INVENTION

### Bleaching Method

The invention aims to meet this need and relates to a cosmetic method for bleaching the hair, comprising at least the steps consisting in:
a) applying a bleaching composition comprising one or more persulfates to at least one lock of hair,
b) exposing the hair impregnated with the bleaching composition to microwave radiation.

The method according to the invention makes it possible to obtain an improved bleaching treatment for the hair. The use of microwave radiation, while the bleaching composition is still present on the hair, provides targeted excitation of the molecules, making it possible to reduce the concentration of the bleaching composition and in particular the concentration of persulfate(s), affording the treatment better efficacy. Thus, for an equivalent result, it is possible to reduce the concentration of persulfates and/or the exposure time.

The use of compositions having pH values lower than those of the known bleaching compositions is also possible.

The use of microwave radiation makes it possible to accelerate the bleaching reaction and therefore to reduce the required leave-on time of the composition.

The durability and the homogeneity of the results obtained from the method according to the invention are very good, and the damage undergone by the hair is reduced and the risks of irritation limited.

Different results are obtained as a function of the duration of exposure to microwave radiation, depending in particular on the base color of the lock of hair.

The duration of the exposure to microwave radiation may vary from 1 second to 30 minutes, better still from 1 second to 10 minutes, even better still from 1 second to 5 minutes, most preferentially from 1 second to 2 minutes.

The method according to the invention may also comprise one or more steps (c) of rinsing the hair, preferably a single rinsing step performed after the step (b) of exposure to microwave radiation.

The method according to the invention may also comprise a step (d) of shaping the hair, comprising in particular the application of a mechanical tension to said one lock of hair, in particular via a mechanical-tension-application device having a reception surface intended to receive the lock of hair, in particular a step of winding the lock of hair on a curler.

In parallel, the method according to the invention can thus contribute to a certain amount of shaping of the hair, and also offers the possibility of performing the durable shaping and bleaching treatment simultaneously.

In this variant, the shaping step (d) is preferably carried out before the step (b) of exposure to microwave radiation.

The method according to the invention may also comprise one or more pre-treatment steps carried out before steps (a) and (b), and/or before or after step (d), if appropriate.

The method according to the invention may also comprise one or more post-treatment steps carried out after step (b), and/or after step (c), if appropriate.

These additional pre-treatment or post-treatment steps may comprise the application of one or more additional compositions other than that present during step (a). The application of these additional compositions may optionally be immediately followed by treatment with microwaves.

In the case in which a shaping step (d) is performed, the pre-treatment step may consist in the application of a composition comprising either one or more fixing film-forming polymers or one or more agents for reducing the disulfide bridges of the keratins.

### Composition

In a preferred embodiment of the invention, the bleaching composition is aqueous, preferably comprising at least 5% by weight of water at ambient temperature (25° C.) and at atmospheric pressure (760 mmHg).

The bleaching composition may result from mixing a plurality of compositions, preferably being the mixture of an anhydrous composition a1 comprising one or more persulfates and an aqueous composition a2, the latter possibly itself being a mixture of at least two compositions.

The mixing of the compositions a1 and a2 may be performed at the time of use of the bleaching composition.

The one or more persulfates included in the bleaching composition are preferably chosen from ammonium persulfate or alkali metal and/or alkaline-earth metal persulfates, such as for example sodium or potassium persulfates.

The bleaching composition used in the method according to the invention can be as described in the patent FR 2 975 291.

In the bleaching composition, the concentration of persulfates may vary from 1% to 60% by weight, better still from 5% to 50% by weight, even better still from 10% to 40% by weight, relative to the total weight of the composition.

If appropriate, the concentration of persulfates in the anhydrous composition a1 may vary from 2% to 80% by weight, better still from 10% to 70% by weight, even better still from 20% to 66% by weight, relative to the total weight of the anhydrous composition a1.

The bleaching composition preferably has a pH greater than 7, preferably ranging from 7.1 to 13, better still from 7.5 to 12 and even better still from 8 to 11.

The bleaching composition preferentially comprises one or more alkaline agents. Said one or more alkaline agents may be contained in the anhydrous composition a1 comprising one or more persulfates and/or in the aqueous composition a2. Said one or more alkaline agents are preferably chosen from metasilicates, alkali metal and/or alkaline-earth metal carbonates, aqueous ammonia, or alkanolamines.

Preferably, the bleaching composition also comprises an additional oxidizing agent, preferably hydrogen peroxide. This hydrogen peroxide may originate from an anhydrous hydrogen peroxide precursor such as urea peroxide that is then present in the anhydrous composition a1 that becomes hydrolyzed in the presence of the aqueous composition a2. Even more preferentially, the hydrogen peroxide may be in the form of an aqueous hydrogen peroxide solution, which is present in the aqueous composition a2.

In a preferred embodiment, the method according to the invention does not use any hair dye.

The cosmetic compositions used in the method according to the invention may also comprise at least one compound chosen from solvents, silicone or non-silicone fatty substances, silicone or non-silicone anionic, cationic, non-ionic or amphoteric surfactants, polymers and in particular anionic, cationic, non-ionic or amphoteric treating, fixing or thickening polymers, agents for breaking disulfide bonds, antidandruff agents, or agents for preventing hair loss.

### Microwave Radiation

The term “microwaves” should be understood as meaning electromagnetic radiation with a frequency ranging from 500 MHz to 300 GHz.

Preferably, the microwave radiation frequency used in the method of the invention ranges from 500 MHz to 300 GHz, better still from 500 MHz to 10 GHZ, even better still from 900 MHz to 5 GHz, most preferentially from 2 GHz to 3 GHz.

The power of the microwaves used during step b) may be less than or equal to 500 W, preferably ranging from 30 to 300 W, better still from 100 to 250 W, even better still from 100 to 200 W or from 100 to 175 W.

Preferably, the surface power density on the hair, induced by the microwave radiation, may range from 0.1 W/cm² to 100 W/cm².

The exposure to microwave radiation may be realized by virtue of a microwave emission antenna, said antenna and at least one part of the lock to be treated preferably being inserted into a microwave-proof chamber.

If appropriate, the chamber, at least during step b), contains the hair to be treated that has been impregnated with the bleaching composition. The term “contain the hair” should be understood as meaning contain the hair over all or part of its length and over a given width. The chamber may cover the hair over a length, for example, of greater than or equal to 5 cm. Thus, a length of at least 5 cm of hair may be treated all at once in the chamber.

Said chamber may be defined at least partially by a casing having a housing, configured to receive at least one part of the hair to be treated and, if appropriate, a tool for shaping the head of hair, and by a cover that pivots relative to the casing between an open position giving access to the housing and a closed position, the microwave emission antenna being disposed inside the housing.

In one variant, the chamber is in the form of a hood.

The chamber may be immobile relative to the hair treated during the emission of the microwaves or mobile relative to the hair, making it possible to treat a greater length of hair.

The chamber may comprise at least one seal of an electrically conductive material, which is, for example, elastically deformable, making it possible to block the microwaves used during step b) while allowing the hair to leave the chamber. The seal may comprise a foam filled with electrically conductive particles, a brush formed from electrically conductive bristles or a comb comprising metal teeth.

In the case in which the chamber is in the form of a hood, the chamber may comprise electromagnetic shielding through which the treated hair may pass. Such shielding makes it possible to treat the user's hair while protecting the user's skull from the microwaves emitted. The electromagnetic shielding is formed, for example, by a grate or a metal grating.

### Assembly

According to another of its aspects, the invention further relates to an assembly for bleaching the hair, comprising at least one bleaching composition comprising one or more persulfates and a device designed to expose the hair to microwave radiation as defined above.

Said device may comprise a microwave emission antenna, said antenna and at least one part of the lock to be treated being in particular inserted into a microwave-proof chamber, as described above.

The microwaves may be generated inside said device designed to expose the hair to microwave radiation or outside by a microwave generator, for example a solid-state generator such as a magnetron. An external microwave generator can supply one or more devices according to the invention, in particular via flexible waveguides.

In one variant, the generator is outside the chamber.

In this case, the generator can be connected to a plurality of antennae, each linked to a device designed to expose the hair to microwave radiation.

Each antenna can be connected to a corresponding high-frequency stage of the generator. A plurality of antennae can be connected to one and the same high-frequency stage of the generator.

In another variant, the generator is inside the chamber.

The microwaves may be emitted by a non-rectilinear antenna, for example of the helical type.

The antenna may be formed from a sufficiently rigid wire, for example with a diameter ranging from 1 mm to 3 mm, so as not to require any holding or support rod for the turns. In one variant, the antenna comprises a flexible wire or a conductive track disposed on a support structure, which is in particular helical. This support structure may be made of any dielectric material, preferably PTFE or glass.

The antenna, when it is helical, preferably has a fixed helical pitch, for example ranging from 4 mm to 5 mm. The radius of the helix is preferably constant, for example ranging from 4 mm to 10 mm. The diameter of the antenna is for example from 8 mm to 10 mm.

The axial extension of the antenna may vary from 50 mm to 80 mm, better still from 65 mm to 75 mm.

The antenna may be disposed in a tube, which is in particular closed at one end. This tube may be made of PTFE or of another dielectric material.

The antenna may also be embedded in a dielectric material, which is preferably flexible, in particular silicone.

Said device designed to expose the hair to microwave radiation may comprise a tool for shaping the head of hair, in particular a tool for applying a mechanical tension, in particular a curler. The term “curler” denotes a tool for winding the hair around the longitudinal axis of the device according to a defined diameter, thus creating a mechanical tension, which is a function of the diameter.

The shaping tool may be configured, if appropriate, so as to place the treated hair flat during the exposure to the microwaves.

The device may comprise a housing that is able to receive the shaping tool. It is possible to coat said housing with a conductive foam that is disposed so as to run over into a hair exit slit. This makes it possible to obtain a good seal of the closure, by virtue of the deformability of the foam at the slit.

If appropriate, the shaping tool and the antenna form a unitary assembly that is introduced into the chamber.

The shaping tool can be made of PTFE or of other dielectric materials, in particular thermoplastics.

The device for implementing the method according to the invention may comprise an audible and/or visible warning system, for example for warning the user of a microwave leak out of the chamber and/or of an excessive temperature inside the chamber.

The device advantageously comprises a safety system for preventing the emission of microwaves while the chamber is not closed and/or in the event of abnormal operation, for example of a microwave leak and/or of excessive temperature.

The device may be configured to control the duration of emission of the microwaves, so as not to reach a duration of treatment that is liable to damage the hair.

The method according to the invention may comprise, before step b), a step of detecting the closure of the chamber. For example, a contactor is actuated when the chamber is closed.

The sending of the microwaves may be conditional on the detection of closure of the chamber. After the microwaves have been sent, the continuation of the microwave emission can be conditional on the absence of the detection of a microwave leak, in particular using a microwave-sensitive sensor placed outside the chamber.

The method according to the invention may also comprise a step of detecting the putting in place of the hair intended to be treated, before step b). This detection step may be performed, for example, by an optical sensor and/or a mechanical feeler. The detection may be done, if appropriate, indirectly, by detecting the putting in place of the shaping tool. The method according to the invention may comprise, for example during step b), a step of measuring the temperature to which the treated hair is subjected. This temperature measurement step may be performed by a thermometer without contact with the hair.

The device may comprise, within the same hand-held piece, the microwave generator and the shaping tool. The term “hand-held piece” denotes a piece manipulated by the user in one hand during the hair treatment.

As conveying means that may be used for conveying the microwaves from the generator to the chamber, mention may be made of waveguides, for example a flexible coaxial cable less than 10 m long, preferably less than 5 m long, less than 5 cm and preferably less than 2 cm in external diameter.

The device may comprise a sensor that is sensitive to a characteristic of the hair, for example the color, the mechanical strength, the surface state or the humidity, and the device may control at least one parameter of the treatment as a function of the characteristic thus detected, for example the microwave energy, the solvent temperature, the duration of the treatment and/or the mechanical stress exerted, if appropriate.

The characteristics set out above for the method apply to the assembly, and vice versa.

### BRIEF DESCRIPTION OF THE DRAWINGS

The invention will be better understood upon reading the following description and examining the accompanying figures. The latter are only presented by way of non-limiting indication of the invention.

FIG. 1 illustrates various steps of an example of a method for bleaching the hair according to the invention,

FIG. 2 is a partial schematic representation of an example of a device designed to implement the method according to the invention,

FIG. 3 shows, open, the hand-held piece of an example of a device designed to implement the method according to the invention,

FIG. 4 shows the hand-held piece in FIG. 3, closed,

FIG. 5 is a view, similar to FIG. 2, of a variant of the device designed to implement the method according to the invention,

FIG. 6 shows a shaping tool, once extracted from the hand-held piece of a variant of the device designed to implement the method according to the invention, and

FIG. 7 is a side view in transverse cross section of a device designed to implement the method according to the invention and comprising the shaping tool in FIG. 6, when the chamber is closed.

### DETAILED DESCRIPTION

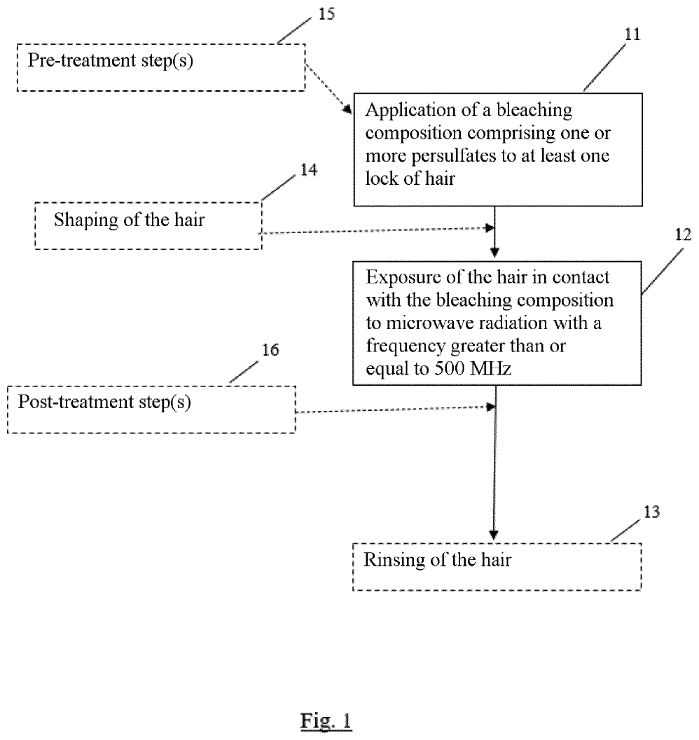

Various steps of an example of a method for bleaching the hair according to the invention are depicted in FIG. 1 and will be described in detail in what follows.

In this example, during a step 11, a bleaching composition comprising one or more persulfates is applied to at least one lock of hair.

In a step 12, the hair in contact with the bleaching composition is exposed to microwave radiation with a frequency greater than or equal to 500 MHz, preferably ranging from 900 MHz to 5 GHz. The duration of the exposure to microwave radiation preferably ranges from 1 second to 1 minute, better still from 2 seconds to 30 seconds. The surface power density on the hair, induced by the microwave radiation, may range from 0.1 W/cm² to 100 W/cm².

The method according to the invention also comprises, in the example described, one or more steps 13 of rinsing the hair, preferably a single step 13 of rinsing the hair, performed after the step 12 of exposure to microwave radiation.

The method according to the invention may also comprise a step 14 of shaping the hair, comprising in particular the application of a mechanical tension to said at least one lock of hair via a mechanical-tension-application device having a reception surface intended to receive the lock of hair, in particular a step of winding the lock of hair on a curler. Preferably and as in the example illustrated, this shaping step 14 is carried out before the step 12 of exposure to microwave radiation.

The method according to the invention may also comprise one or more pre-treatment steps 15 carried out before steps 11 and 12, comprising for example the application of one or more additional compositions other than that used during step 11.

One or more post-treatment steps 16 may be carried out after step 12 and/or after step 13, comprising for example the application of one or more additional compositions other than that used during step 11.

Preferably and as in the example described, the bleaching composition has a pH greater than 7 and is aqueous, comprising for example at least 5% by weight of water at ambient temperature and at atmospheric pressure. The bleaching composition may be a mixture of a plurality of compositions, being in the example in question the mixture of an anhydrous composition comprising one or more persulfates a1 and an aqueous composition a2, the mixing of the compositions being performed at the time of use of the bleaching composition. The one or more persulfates included in the bleaching composition and their concentration are as described above.

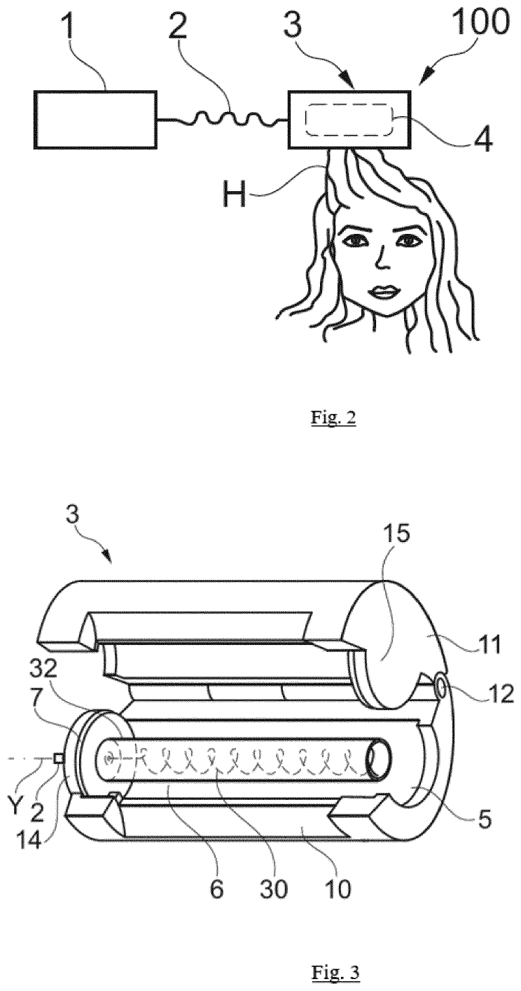

FIG. 2 shows a device 100 designed for the implementation of the method according to the invention, comprising a hand-held piece 3 connected to a base station 1, comprising a microwave generator, via a flexible waveguide 2, constituted by a shielded cable. The hand-held piece 3 is intended to be manipulated by the user in order to be placed in proximity to the person's head so as to treat at least one lock of hair H, as illustrated. The hand-held piece 3 can be produced in various ways and defines a microwave-proof treatment chamber 4.

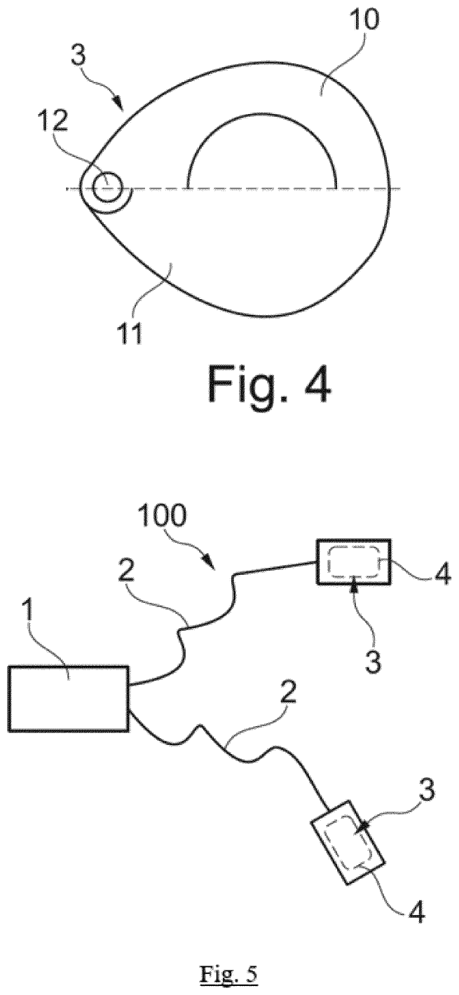

In the example shown in FIG. 3, the hand-held piece 3 comprises a casing 10, defining a housing 6, on which a cover 11 is articulated by means of a pin 12. The hand-held piece is shown closed in FIG. 4. The flexible waveguide 2 has been represented only very partially in FIG. 3 and the following figures.

The casing 10 bears an antenna 30 that is, in the example shown, helical in shape, with a longitudinal axis 7.

The antenna 30 is, in the example in question, made up of a helical metal wire, disposed in a tube 32 of a dielectric material, for example silicone or PTFE.

The antenna 30 is supported at an axial end of the casing 10 by a plate 14 of the casing 10, which has a semicircular outline.

The cover 11 has a similar plate 15 on the opposite side, closing the opening 5 of the housing 6, via which a shaping tool may be introduced into the hand-held piece 3.

The interior surface of the housing 6 is defined by an electrically conductive coating 7 that makes it possible to reflect the microwaves and makes the chamber 4 microwave-proof. This coating 7 can be formed from a conductive foam, which is for example semi-cylindrical in shape on the cover and the casing, in the form of a disk for the plate 15 and of a ring for the plate 14.

The casing 10 and the cover 11 define an exit slit 19 for the hair, located at the bottom of an indentation 18, on the side opposite the pin 12 of the hinge.

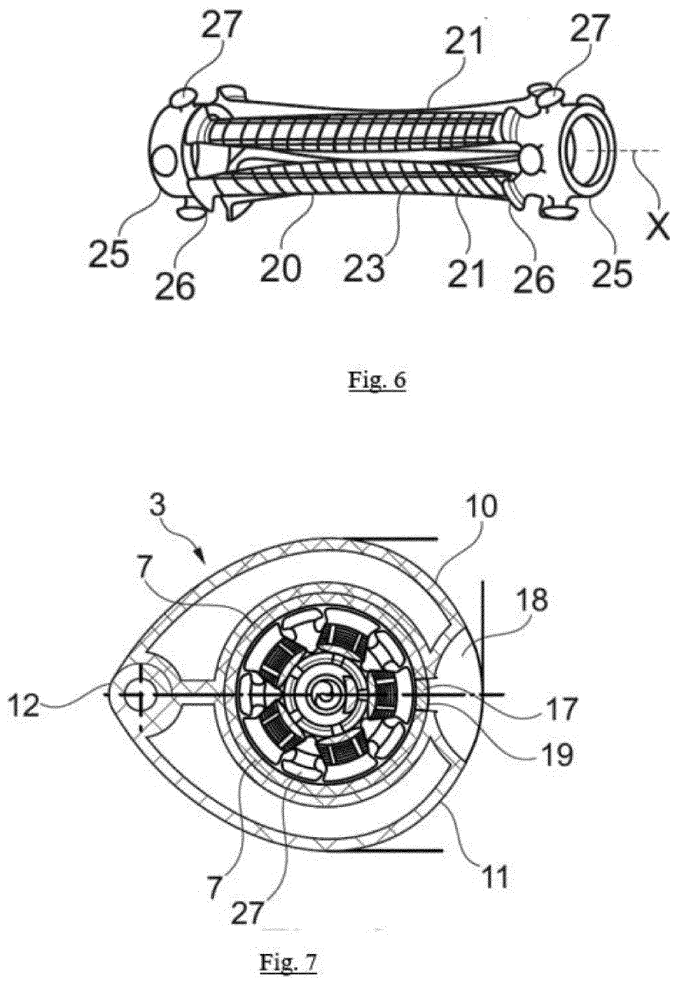

The coating 7 preferably protrudes into the slit 19, as can be seen in FIG. 7, so as to produce a good closure seal despite the hair passing through. The edge of the coating can thus go slightly beyond, at 17, the edge of the body of the cover and that of the casing, closing the slit 19.

The hand-held piece 3 comprises a member for locking the cover in the closed position, not represented, and also an opening detector.

A temperature detector may be present in the chamber 4, for example on an interior surface thereof, in order to measure the temperature of the treated lock of hair, and a control system, for example a micromethodor system, may make it possible to interrupt or modify the microwave emission in the event of a detected temperature above a predefined threshold.

A plurality of hand-held pieces 3 may be connected to one and the same generator 1, as shown in FIG. 5.

### Particular Variant

In one particular embodiment, the casing 10 defines a housing 6 for receiving a shaping tool constituted, in the example in question, by a curler 20, shown in FIG. 6. The presence of the tube 32, shown in FIG. 3, in which the wire of the antenna 30 is disposed, makes the insertion of the latter into the curler 20 easier.

Said curler comprises a plurality of branches 21 that define a surface for receiving the hair H, of width L, measured along the longitudinal axis X of the curler 20.

The axis X is, in this example, parallel to the axis of rotation of the cover 11 and coincident with the longitudinal axis 7 of the antenna 30.

In the example in question, the curler 20 comprises four branches 21, which are slightly curved toward the axis X and pass by a minimum distance to the axis X at about mid-length.

The branches 21 can have ribs 23 oriented perpendicular to the axis X for assisting in holding the lock spread out on the branches 21, while preferably occupying the entire width L as uniformly as possible.

The branches 21 connect at their ends to annular parts 25.

The curler 20 has teeth 26 at the ends of the branches 21, which laterally delimit the surface for receiving the hair.

The curler 20 can be made of any dielectric material, preferably of PTFE.

The curler 20 preferably has a shape that exhibits symmetry of revolution relative to a median plane of symmetry perpendicular to the axis X.

The curler 20 comprises blocks 27 with a widened head, which connect at their base to the corresponding annular parts 25, which press against the coating 7, as shown in FIG. 7, so as to hold the curler centered in the housing 6.

The blocks 27 are for example located between the teeth 26, as shown.

The width W of the exit slit 19 for the hair can be slightly greater than L, the length of the housing 6 being substantially equal to the length of the curler 20, such that the latter is immobilized both axially and radially in the housing 6, to within a slight amount of play.

In order to use the hand-held piece 3, a lock of hair impregnated with bleaching composition is wound on the curler 20, then the latter is disposed inside the casing 10 by moving it laterally relative to said casing, while introducing it via the opening 5. The cover 11 is then closed again, and the hair exits via the slit 19. Once the microwave exposure has been performed, the lock of hair is extracted by performing the operations in the reverse order.

Numerous modifications can be made to the methods and devices that have just been described, without departing from the scope of the present invention.

The invention is not limited to a particular type of shaping tool, or to a particular type of microwave emission antenna.

1. A cosmetic method for bleaching the hair, comprising at least the steps consisting in:
a) applying a bleaching composition comprising one or more persulfates to at least one lock of hair,
exposing the hair impregnated with the bleaching composition to microwave radiation.

2. The method according to claim 1, wherein the microwave radiation frequency used in the method of the invention ranges from 500 MHz to 300 GHz, better still from 500 MHz to 10 GHz, even better still from 900 MHz to 5 GHZ, most preferentially from 2 GHz to 3 GHz.

3. The method according to claim 1, wherein the bleaching composition is aqueous, comprising in particular at least 5% by weight of water at ambient temperature and at atmospheric pressure.

4. The method according to claim 1, wherein the bleaching composition results from mixing a plurality of compositions, being in particular the mixture of an anhydrous composition comprising one or more persulfates a1 and an aqueous composition a2, the mixing of the compositions being in particular performed at the time of use of the bleaching composition.

5. The method according to claim 1, wherein the one or more persulfates included in the bleaching composition are chosen from ammonium persulfate or alkali metal and/or alkaline-earth metal persulfates, such as in particular sodium or potassium persulfates.

6. The method according to claim 1, wherein, in the bleaching composition, the concentration of persulfates varies from 1% to 60% by weight, better still from 5% to 50% by weight, even better still from 10% to 40% by weight, relative to the total weight of the composition.

7. The method according to claim 1, wherein the bleaching composition has a pH greater than 7, ranging in particular from 7.1 to 13, better still from 7.5 to 12 and even better still from 8 to 11.

8. The method according to claim 1, wherein the bleaching composition comprises one or more alkaline agents, chosen in particular from metasilicates, alkali metal and/or alkaline-earth metal carbonates, aqueous ammonia, or alkanolamines.

9. The method according to claim 1, wherein the bleaching composition comprises an additional oxidizing agent, preferably hydrogen peroxide.

10. The method according to claim 1, wherein the exposure to microwave radiation is realized by virtue of a microwave emission antenna, said antenna and at least one part of the lock to be treated being in particular inserted into a microwave-proof chamber.

11. The method according to claim 1, wherein the surface power density on the hair, induced by the microwave radiation, varies from 0.1 W/cm² to 100 W/cm².

12. The method according to claim 1, wherein the duration of the exposure to microwave radiation varies from 1 second to 30 minutes, better still from 1 second to 10 minutes, even better still from 1 second to 5 minutes, most preferentially from 1 second to 2 minutes.

13. The method according to claim 1, also comprising a step (d) of shaping the hair, comprising in particular the application of a mechanical tension to said at least one lock of hair, in particular via a mechanical-tension-application device having a reception surface intended to receive the lock of hair, in particular a step of winding the lock of hair on a curler, the shaping step d preferably being carried out before the step b of exposure to microwave radiation.

14. Assembly for bleaching the hair, comprising at least one bleaching composition comprising one or more persulfates and a device designed to expose the hair to microwave radiation with a frequency ranging from 500 MHz to 300 GHz, better still from 500 MHz to 10 GHz, even better still from 900 MHz to 5 GHz, most preferentially from 2 GHz to 3 GHz.

15. The assembly according to claim 14, wherein the device comprises a microwave emission antenna, said antenna and at least one part of the lock to be treated being in particular inserted into a microwave-proof chamber.
